(19) United States
(12) Patent Application Publication
(10) Pub. No.: US 2003/0196666 A1
(43) Pub. Date: Oct. 23, 2003

(54) Safe t-Tube

(76) Inventor: Ralph Leonard, New Haven, CT (US)

Correspondence Address: Ralph Leonard, Apartment 400, 1145 Chapel Street, New Haven, CT, 06510, US

(21) Appl. No.: 10/127,433

(22) Filed: Apr. 22, 2002

(51) Int. Cl. A61M 16/00

(52) U.S. Cl. 128/207.14

(57) ABSTRACT

This invention is a device that is intended to address the problem of patient asphyxiation of intubated patients resulting from a disconnection of the endotracheal tube and ventilator tubing. The proposed device is a t-shaped piece of tubing that is firmly attached such that its two ends which are not covered (the so-called parallel cylinder) are connected to the endotracheal tube and ventilator tubing, forming a conduit between these tubes. Running perpendicular to this parallel cylinder is the ‘t’ component of the tubing on which are mounted impermeable discs that serve as spring-loaded valves that open when the air pressure within the ventilation system exceeds the desired level, thus allowing air to flow into the lower pressure surrounding environment and dissipate the pressure within the ventilation system.

### CROSS-REFERENCE TO RELATED APPLICATIONS

To the best of my knowledge, there are no prior patents which describe the process or a similar mechanism to address the problem that is the content of this patent.

### STATEMENT REGARDING FEDERALLY SPONSORED RESEARCH OR DEVELOPMENT

There were no direct or indirect federally sponsored research money or support given for the development of this application or idea.

### REFERENCE TO A MICROFICHE APPENDIX

Does not apply.

### BACKGROUND OF THE INVENTION

Definitions: For the purposes of the rest of this application, I will use the following terms as defined here. 1) Distal: a medical expression used to indicate a point of interest is farther from the patient than what it is being referenced to 2) Endotracheal tube: a tubing that is inserted into the patient's trachea (“wind-pipe”) and secured such that one end stays in the trachea and the other end juts from the mouth. A variant of this is a nasotracheal tube which is inserted such that the external end projects via the nose. There are numerous models and sizes, but for the purposes of this patent application all versions and models will be represented by the term ‘endotracheal tube.’ 3) Proximal. a term used to indicate a point of interest is closer to the patient than what it is being referenced to. 4) Parallel cylinder: That cylinder of the Safe t-Tube whose ends connect with ventilator tubing and an endotracheal tube. 5) Perpendicular cylinder: That cylinder of the Safe t-Tube whose ends are covered with discs that can function as valves. 6) Ventilator: a mechanical device external to the patient which provides artificial respiration such that a patient receives a specified amount of oxygen and air volume or pressure at a specified rate. 7) Ventilator tubing: Tubing that connects the ventilator and endotracheal tube. 8) ventilation system: the space which comprises the net total of the following: patient's lungs, inside of the endotracheal tube, inside of the ventilator tubing and inside of the ventilator which is exposed to air going to or coming from the patient. 9) opening air pressure: the pressure in the ventilation system at which the discs of the Safe t-Tube move from the closed to the open position.

The problem which this invention seeks to address is asphyxiation that results when ventilator tubing disconnects from an endotracheal tube. Such a seemingly avoidable event occurs, in part, by design. When patients are attached to a ventilator there are a number of potential complications they may suffer, including “barotrauma” which is a type of lung injury resulting from high air pressure within the ventilation system. Such sudden high ventilation system pressure may be the result of several causes such as ventilator malfunction or a sudden cough.

The current methods of avoiding accidental barotraumas are two fold: 1) almost all modern ventilators have alarms which the technician may set such that an attendant is notified when pressure within ventilation system is higher than a preset safe level and 2) the seal that joins the ventilator tubing with the endotracheal tube is fragile such that the ventilator tubing will disconnect from the endotracheal tube which then open the otherwise closed ventilation system and quickly dissipates the high pressure.

This second method described above, is an attempt to be fool-proof so that if the ventilator fails in either its administration or measurement of pressure, the seal of the endotracheal tube and ventilator tubing will break at a given air pressure. This practice is based on the assumption that modem ventilators measure the volume or pressure of air returning from the patient and when the seal is broken one or both of those abnormal measures would trigger an alarm. While the benevolent intent is apparent, the reality is quite different and patients asphyxiate for two reasons: 1) after the seal is broken the ventilator alarms are not emitted either because of human error in setting their levels or machine malfunction 2) in busy intensive care units the alarms indicating a disconnected seal may not be heard by staff who may be attending to another patient or can not hear the alarms for other reasons.

### BRIEF SUMMARY OF THE INVENTION

This invention is a device that is intended to address the aforementioned problem of patient asphyxiation resulting from a disconnection of the endotracheal tube and ventilator tubing. The proposed device is a t-shaped piece of tubing that is firmly attached such that its two ends which are not covered (the so-called parallel cylinder) are connected to the endotracheal tube and ventilator tubing, forming a conduit between these tubes. Running perpendicular to this parallel cylinder is the ‘t’ component of the tubing which has impermeable discs that serve as spring-loaded valves that permit air to escape when the pressure within the closed ventilation system exceeds that of the spring, but when the air pressure is less than the spring's pressure on the disc the discs remain close and thus there is no inward flow of air into the ventilation system.

There are many possible variations of this invention with regard to spring force (which would control the pressure at which the valves would open), tubing size and material composition. However, these are minor variations and can be subsumed within the conceptual extent of this patent application.

### BRIEF DESCRIPTION OF THE SEVERAL VIEWS OF THE DRAWING

Please note: 1) none of these figures are drawn to specified scale 2) pairs of figures which share the same prefix number (e.g., 1A and 1B, 2A and 2B) have items labeled just once to avoid clutter.

FIGS. 1-5 depict the problem and application of the Safe t-Tube in a number of views.

FIG. 1A: schematic illustrating a patient, endotracheal tube (with just the external component visible), ventilator tubing and ventilator as they may be arranged in the modern, standard practice.

FIG. 1B: schematic illustrating how the modern, standard practice (shown in FIG. 1A) reacts to a high pressure episode within the ventilator system such that the endotracheal tube and ventilator tubing disconnect.

FIG. 2A: schematic illustrating how the Safe t-Tube is placed in-line with (i.e., such that it serves as a conduit) the endotracheal tube and ventilator tubing.

FIG. 2B: schematic illustrating how the discs on the perpendicular cylinder of the Safe t-Tube act as valves that open during a high pressure episode within the ventilator system.

FIG. 3A: proportioned drawing of the Safe t-Tube from above perspective (i.e., what one might see if looking at its top) with the discs on the perpendicular cylinder closed.

FIG. 3B: proportioned drawing of the Safe t-Tube from above perspective (i.e., what one might see if looking at its top) with the discs on the perpendicular cylinder open.

FIG. 4A: proportional drawing of the Safe t-Tube as it would be seen if one were looking into the parallel cylinder with the discs on the perpendicular cylinder closed.

FIG. 4B: proportional drawing of the Safe t-Tube as it would be seen if one were looking into the parallel cylinder with the discs on the perpendicular cylinder open.

FIG. 5A: proportional drawing of the Safe t-Tube as it would be seen if one were looking into the perpendicular cylinder with the discs on the perpendicular cylinder closed.

FIG. 5B: proportional drawing of the Safe t-Tube as it would be seen if one were looking into the perpendicular cylinder with the discs on the perpendicular cylinder open.

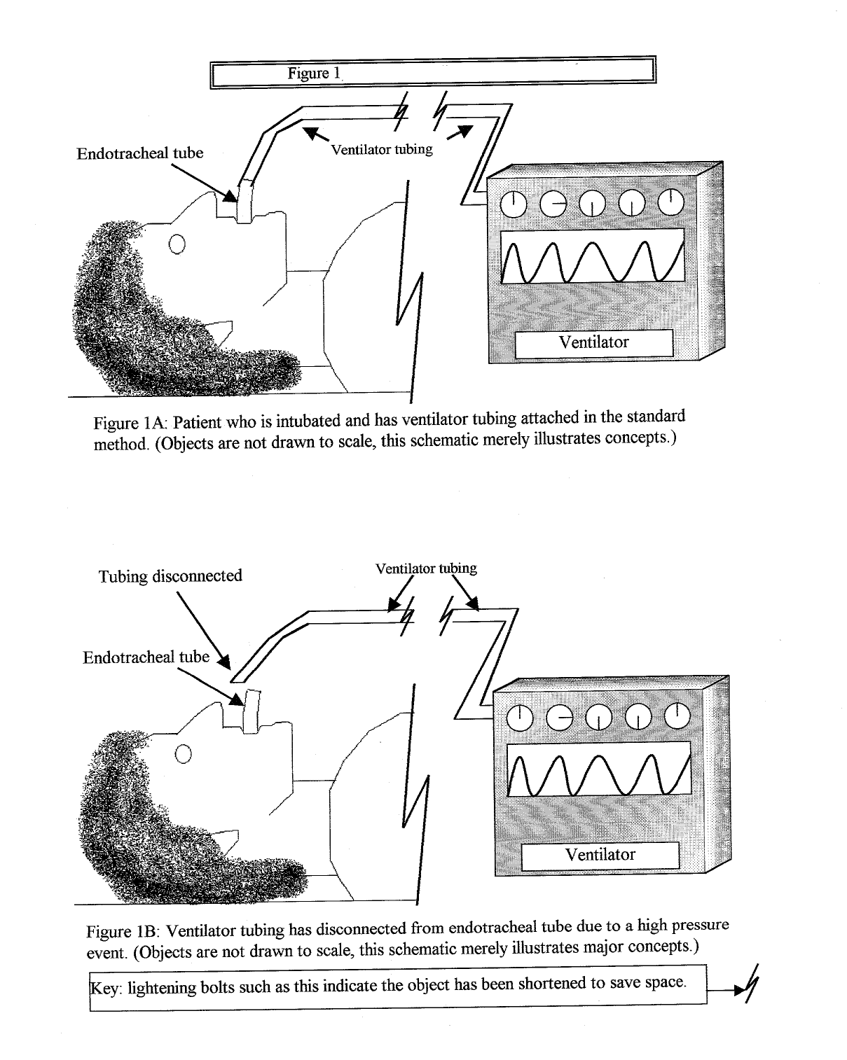

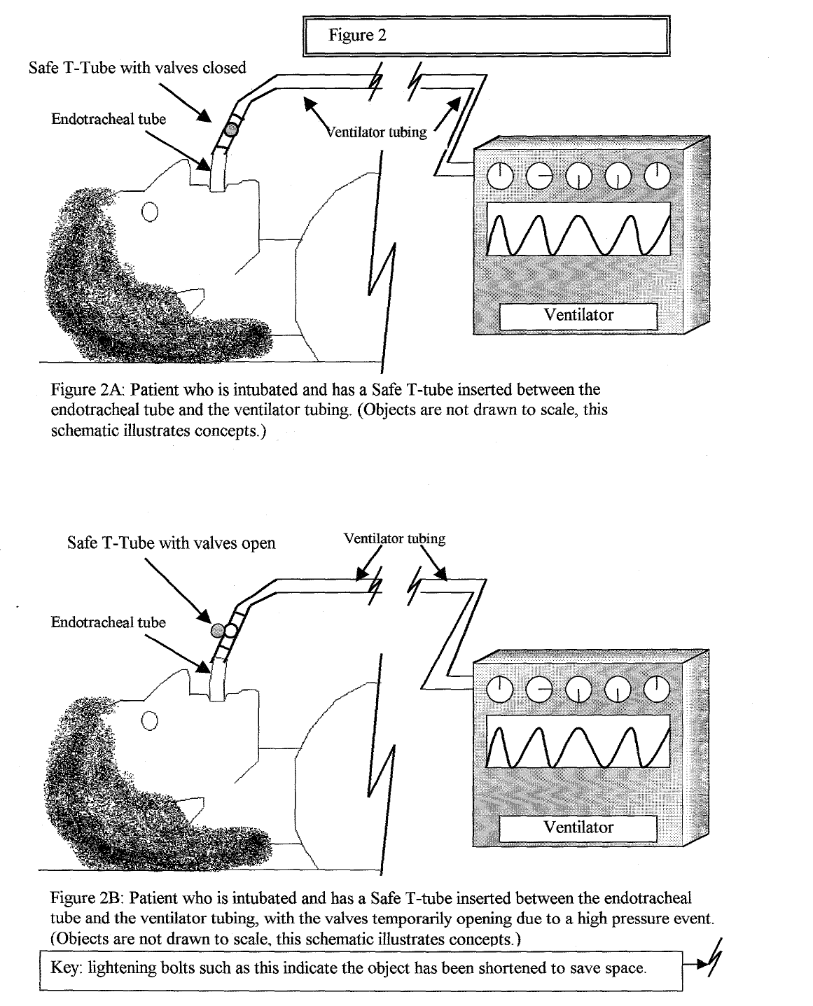

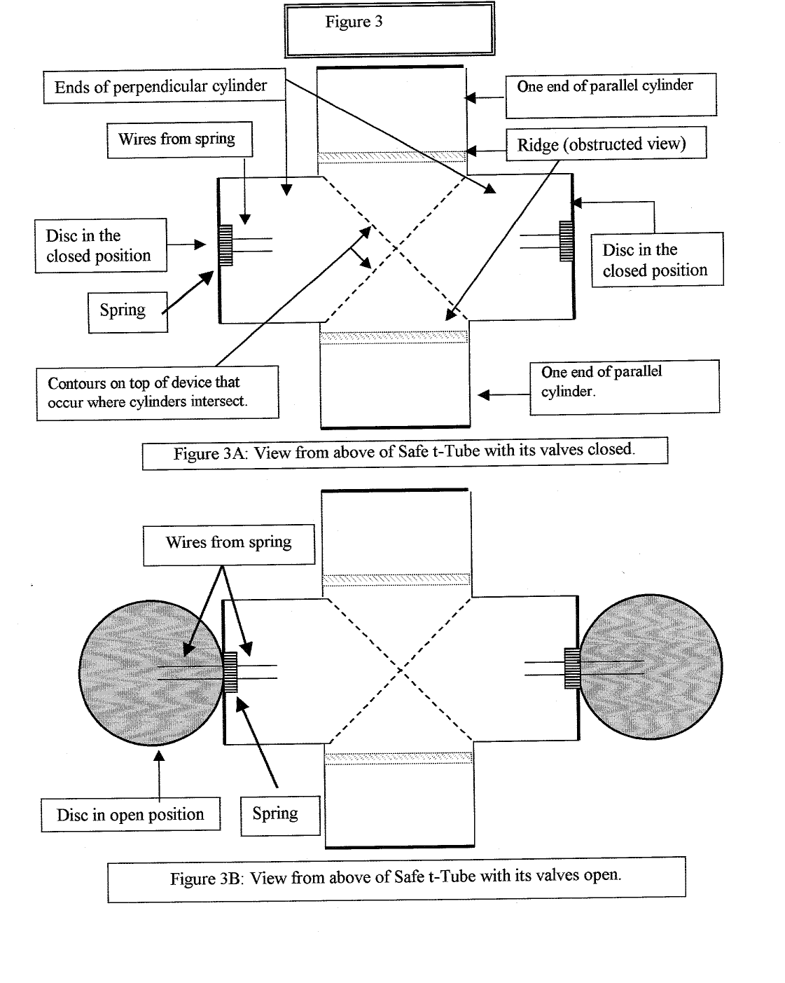

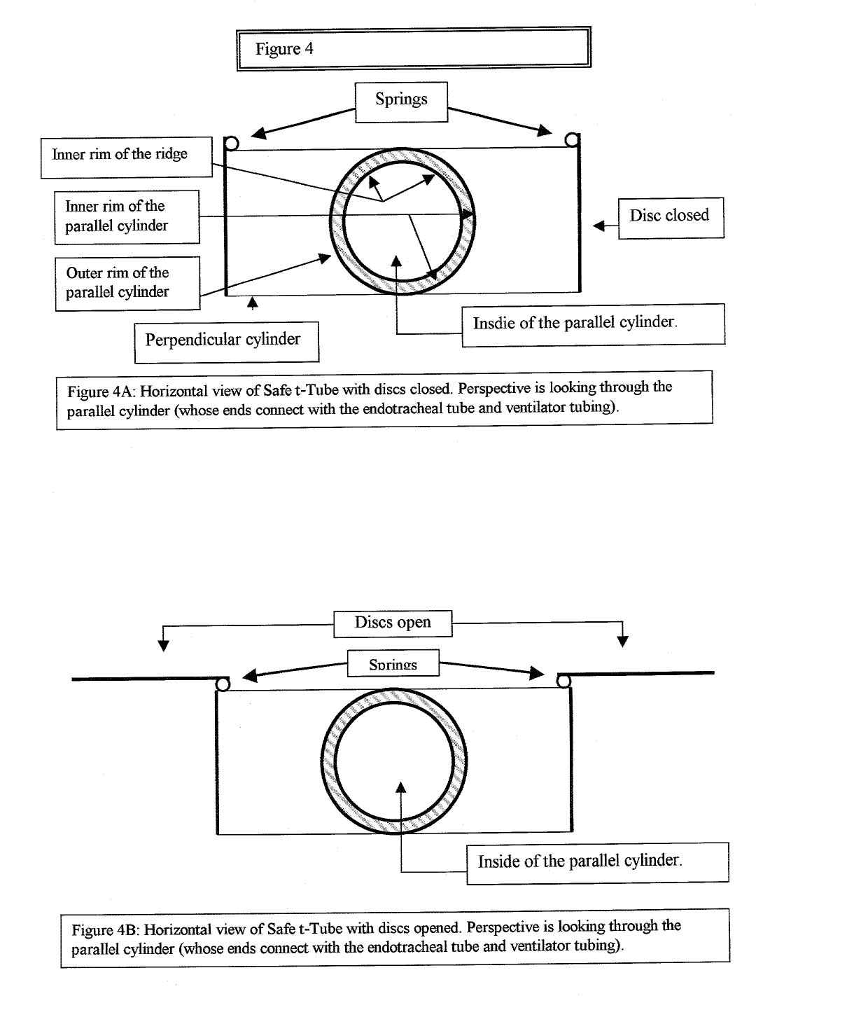

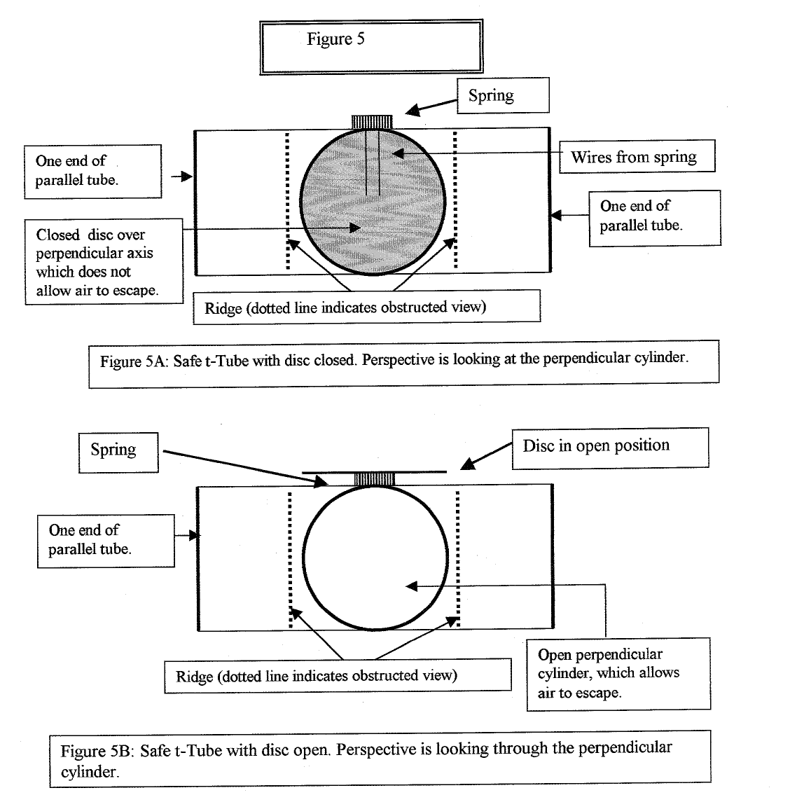

### DETAILED DESCRIPTION OF THE INVENTION

This device can be conceptualized as two cylinders of equal length and diameter intersecting at right angles in their mid sections. When attached, one cylinder may be further described as running parallel and in-line with the endotracheal tube and ventilator tubing of a mechanical ventilation system (called the parallel cylinder); the other cylinder may be further described as running perpendicular to the endotracheal tube and ventilator tubing (called the perpendicular cylinder).

The internal diameter of the parallel cylinders are greater than the external diameter of the endotracheal tube and ventilator tubing such that these latter two may be inserted inside the parallel cylinder.

The interior of the parallel cylinder has two ridges projecting inward located just beyond the mid-section of where the perpendicular and parallel tubes intersect. The height of the rims is at least two millimeters and tubing inserted into the parallel cylinders (either the endotracheal tube or ventilator tubing) may abut against but not project past these rims such that the is never any obstruction inadvertently made by external tubing that projects within the space parallel to the perpendicular cylinder.

Inserted tubes (either the endotracheal tube or ventilator tubing) are inserted into the ends of a parallel cylinder on a version of the Safe t-Tube that has diameters of appropriate size such that the fit is snug; these inserted tubes may be further affixed to the parallel cylinder of the Safe t-Tube with external apparatus such as tape or other devices.

The device has a bottom and top, the latter which is defined by the presence of springs affixed to the edge of the perpendicular cylinder ends. Two discs of diameter slightly larger than that of the perpendicular cylinder are permanently attached to wires in turn part of a spring that is in turn attached to the top of the perpendicular cylinder. The discs are impermeable to water and air and when in the closed position abut against the ends of the perpendicular cylinder and cover its entire diameter such that there is no communication of gases or fluids between the closed ventilation system and surrounding environment under normal ventilation system pressure. At or above a pre-specified ventilation system air pressure the discs are will open and allow air to move across a temporarily created gradient such that it flows from the ventilation system to the surrounding environment. Once the pressure gradient between the internal ventilation system and the environment is less than the pressure exerted by discs, the discs will resume the closed position.

The pressure at which the discs open is determined by the force stored in their springs, the surface area and mass of the discs. Since various pressures may be considered acceptable depending upon the clinical circumstances (e.g., conditions such as Acute Respiratory Distress Syndrome are associated with higher pressures than those commonly seen), this device would be available in numerous versions of spring force such that discs would open only if the ventilation system pressure exceeded the desired amount.

The size of the device and its components may vary depending upon the costs, needs and technology available and are covered by this patent. The diameter of endotracheal tubes and ventilator tubing vary given the range of pediatric to adult patients who are treated. Thus, the device would be available in multiple cylinder diameters. Likewise, while the proposed version of the device has equal diameter in both ends of the parallel cylinder, this does not need to be the case and depending upon the endotracheal tube and ventilator tubing varieties may be made to more closely approximate their sizes where each is to be inserted, respectively.

The material composition of the device may also vary depending upon the choice of the manufacturer and regulatory restrictions. However, the device must be rigid such that it can not be easily compressed by intent or accident. The proposed material of the cylinders and discs is disposable plastic such that the unit is not re-usable and less likely to transfer contagious organisms. The proposed spring composition is stainless steel but the wire diameter and number of revolutions would be determined by the desired opening pressure limit and disc surface area (the latter based upon the diameter of the perpendicular ends). The means by which the spring attaches to the disc and ends of the perpendicular cylinder may be varied and is also covered by this patent, but a proposed method is with permanent, none-toxic, water-proof glue.

This patent also covers different manufacturing process if the end product is as described here, but the preferred means is to use a mold such that the two cylinders are in fact just one unit rather than the joining of two separately made cylinders.

1. I claim as my invention the device which allows for rapid release of high ventilation system pressure while maintaining an in-line connection of endotracheal tube and ventilator tubing, here onwards called the Safe t-Tube. The Safe t-Tube can be conceptualized as comprising two cylinders of equal length and diameter intersecting at right angles in their mid sections. When attached, one cylinder may be further described as running parallel and in-line with the endotracheal tube and ventilator tubing of a mechanical ventilation system (called the parallel cylinder); the other cylinder may be further described as running perpendicular to the endotracheal tube and ventilator tubing (called the perpendicular cylinder).
A. I claim as my invention that the internal diameter of the parallel cylinders of the Safe t-Tube are greater than the external diameter of the endotracheal tube and ventilator tubing such that these latter two may be inserted inside the parallel cylinder.
B. The interior of the parallel cylinder has two ridges located just beyond the intersection of the parallel and perpendicular cylinders such that inserted tubing may never occupy (and thus impede) the space within the perpendicular cylinder. The height of the rims is at least two millimeters and tubing inserted into the parallel cylinders (either the endotracheal tube or ventilator tubing) may abut against but not project past these rims.
C. The patent also covers this device even if the final marketing or trade-name differs from that of the Safe t-Tube.
D. The absolute, relative and proportional size of the device and its components (e.g., discs, springs, spring force) may vary depending upon the costs, needs and technology available and are covered by this patent. The diameter of endotracheal tubes and ventilator tubing vary given the range of pediatric to adult patients who are treated. Thus, the device would be available in multiple cylinder diameters. While the proposed version of the device has equal diameter in both ends of the parallel cylinder, this does not need to be the case and depending upon the endotracheal tube and ventilator tubing varieties may be made to more closely approximate their sizes where each is to be inserted, respectively.
E. The material composition of the device may also vary depending upon the choice of the manufacturer and regulatory restrictions. However, the device must be rigid such that it can not be easily compressed by intent or accident. The proposed material of the cylinders is disposable plastic such that the unit is not re-usable and less likely to transmit organisms.
F. I claim as my invention that the manufacturing process is irrelevant and versions produced by numerous methods are also covered by this patent if their final form is that described herein. The preferred means of manufacture is to use a mold such that the two cylinders are a homogenous unit rather than the joining of two separately made cylinders.

2. I claim as my invention that: the device has a bottom and top, the latter which is defined by the presence of springs affixed to the edge of the perpendicular cylinder ends and there are two discs of diameter slightly larger than that of the perpendicular cylinder which are permanently attached to wires in turn part of a spring that is in turn attached to the top of the perpendicular cylinder.
A. The discs are impermeable to water and air and when in the closed position abut against the ends of the perpendicular cylinder such that there is no communication of gases or fluids below the desired opening-pressure limit. At or above a pre-specified ventilation system pressure the discs are pushed open and allow external air movement from the high pressure internal ventilation system to the lower pressure environment. Once the pressure gradient between the internal ventilation system and the environment is less than the pressure exerted by the springs, the discs will resume the closed position.
B. The pressure at which a disc opens is determined by the surface area of the disc, its mass and the force stored in its springs, however the preferred variant among these three is spring force. Since various pressures may be considered acceptable depending upon the clinical circumstances (e.g., conditions such as ARDS are associated with higher pressures than those commonly seen), this device would be available in numerous versions of spring force and opening air pressure limits.
C. The material composition of the device may also vary depending upon the choice of the manufacturer and regulatory restrictions. The proposed material of the discs is disposable plastic such that the unit is not re-usable and less likely to transmit contagious organisms.
D. The proposed spring composition is stainless steel but the wire diameter and number of revolutions would be determined by the desired opening pressure limit and disc surface area (the latter based upon the device's size).
E. The means by which the spring attaches to the disc and ends of the perpendicular cylinder may be varied and is also covered by this patent; a proposed method is with permanent, none-toxic, water-proof glue.
